(19) United States
(12) Patent Application Publication
(10) Pub. No.: US 2021/0163439 A1
(43) Pub. Date: Jun. 3, 2021

(54) PROCESS FOR PREPARING CARBONATES BY ADDITION OF CO2 WITH AN EPOXIDE

(71) Applicant: EVONIK OPERATIONS GMBH, Essen (DE)

(72) Inventors: Marcel TRESKOW, Darmstadt (DE); Günther GRÄFF, Bensheim (DE); Maik CASPARI, Alsbach-Haehnlein (DE); Thorben SCHÜTZ, Alsbach-Hähnlein (DE); Steffen KRILL, Mühltal (DE); Tim BLEITH, Mainz (DE)

(73) Assignee: EVONIK OPERATIONS GMBH, Essen (DE)

(21) Appl. No.: 16/973,995

(22) PCT Filed: Jun. 7, 2019
(86) PCT No.: PCT/EP2019/064911

§ 371 Date: Dec. 10, 2020

(30) Foreign Application Priority Data
Jun. 11, 2018 (EP) 18176920.9

(51) Int. Cl. C07D 317/38; B01J 31/02

(52) U.S. Cl. CPC C07D 317/38; B01J 31/0268

(57) ABSTRACT

The invention relates to a process for preparing cyclic organic carbonates, characterized in that an epoxide is initially charged in the presence of CO₂ and then a catalyst is added.

### FIELD OF THE INVENTION

The invention relates to a process for preparing cyclic organic carbonates, especially glycerol carbonate (meth)acrylates, by means of CO₂ insertion.

### PRIOR ART

EP1894922 describes a process for preparing glycerol carbonate esters. This document describes the crossed transesterification of MMA with glycerol carbonate acetate to give methyl acetate and glycerol carbonate methacrylate. The process requires complex distillation steps, a subsequent neutralization and subsequent workup by means of phase separation. The yield is only 87%. Moreover, only 67% product (glycerol carbonate methacrylate) and still 27% glycerol carbonate acetate are present in the product mixture.

Büttner et al. (ChemCatChem, 2015, vol. 7, p. 459-467) describe the synthesis of various bifunctional organocatalysts based on ammonium salts and the use thereof. In the reaction of 1,2-butylene oxide with CO₂. The conversion is effected at 45° C. and 1.0 MPa over 18 hours.

Werner et al. (ChemSuSChem, 2014, vol. 7, p. 3268-3271) describe a method of reacting 1,2-butylene oxide with CO₂ in the presence of tri-n-butyl(2-hydroxyethyl)phosphonium iodide.

Problem and solution in the preparation of glycerol carbonate methacrylate by CO₂ insertion, ideal yields of 99% are possible on the gram scale. On larger scales, however, ail known processes lose selectivity, and there is formation of by-products and discoloration. By-products are critical especially when they are crosslinkers. In order to enable use of glycerol carbonate methacrylate, the crosslinker content has to be at a minimum. A crosslinker content of greater than 1% is prohibitive to any application, in addition, in the preparation of carbonates by the present route, a high pressure is generally required. In conventional production plants, a high pressure is not possible, and so special high-pressure tanks would be required for preparation.

The sequence in the case of Werner et al. in larger batches exceeding the gram scale leads to unwanted by-products.

On a small scale, in the mmol range, as in the case of Werner, the reaction system can be provided sufficiently rapidly with the CO₂. By Werner's method, there is a distinct increase in by-products with increasing batch size.

For industrial-scale implementation, this makes it impossible to prepare the product with sufficient purity by the process described above.

Moreover, in the case of Werner et al., there is disadvantageous exothermicity of the process. Even without further energy supply, a batch on a larger scale heats up to temperatures well above 75° C., the final temperature being determined primarily by the batch size. Above 85° C., however, a side reaction that again leads to formation of crosslinkers commences, which surprisingly does not seem to be relevant on a small scale (a few grams) since the literature conducts this experiment at 90° C.

Werner et al. describe, in ChemSusCem, 2014, vol. 7, #12, p. 3268-3271, the use of a bromide-containing catalyst. However, owing to its low activity, this is classified as being not very suitable, and a more active iodide-based catalyst is preferred. From a commercial point of view, however, the more active catalyst is additionally unsuitable since the reactants needed for the preparation of the catalyst, in the case of iodoethanol, are not available in commercial amounts but exclusively as fine chemicals. This distinctly increases the cost of a synthesis (both of the product and of the catalyst) on a commercial scale.

The problem addressed was that of developing a process which overcomes the above-described disadvantages.

The problem is solved by a process for preparing cyclic organic carbonates, characterized in that an epoxide is initially charged in the presence of CO₂ and then a catalyst is added.

More particularly, a process for preparing glycerol carbonate (meth)acrylates is claimed, characterized in that a glycidyl (meth)acrylate is initially charged in the presence of CO₂ and then the catalyst is added.

It has been found that, surprisingly, the sequence in which the constituents of the reaction encounter one another is of crucial importance. The catalyst is already active at room temperature and, in the absence of CO₂, leads to formation of crosslinkers and to a yellow color of the product.

The process regime according to the invention therefore stipulates that the catalyst should only be supplied to the reactor mixture after the CO₂.

It has been found that a reaction at a temperature below 90° C. can be conducted with distinctly lower by-product formation.

According to the invention, therefore, the reaction is effected at temperatures between 10 and 85° C., preferably between 15 and 80° C., more preferably between 20 and 75° C.

It has been found that it is particularly advantageous when the temperature is increased stepwise. There is typically an increase by 10° C. every 15 min. Optionally, the temperature is increased even more slowly, for example stepwise from 70 to 85° C. within three hours.

The process according to the invention is particularly advantageous when the reaction scale is greater than 5 mol.

The present process is concerned with the preparation of carbonates by CO₂ insertion into epoxides at pressures between 1 and 10 bar, preferably between 2 and 8 bar, more preferably between 3 and 7 bar and most preferably at 5 bar. Standard steel tanks are designed for pressures of −1 to +6 bar, and so, at a synthesis pressure of 5 bar, performance is also possible in conventional equipment. Existing processes with low pressures have very long reaction times that oppose production on a commercial scale.

The notation “(meth)acrylate” here means both methacrylate, for example methyl methacrylate, ethyl methacrylate, etc., and acrylate, for example methyl acrylate, ethyl acrylate, etc., and mixtures of the two.

Reactants

Suitable reactants are a multitude of epoxides. Suitable examples are propene oxide, 1-butene oxide, octene oxide, 3-chloro-1-propene oxide, glycidyl (meth)acrylate, cyclohexene oxide, isobutene oxide, 2-butene oxides, styrene oxide, cyclopentene oxide, ethene oxide and hexene oxide, and mixtures thereof.

Particularly suitable epoxides are selected from the group of glycidyl methacrylate, isobutene oxide, 2-butene oxides, styrene oxide, cyclopentene oxide, ethene oxide and hexene oxide.

Catalysts Suitable catalysts may be selected from the group of the halide and pseudohalide salts of elements of main group 5.

Particularly suitable catalysts are selected from the group of Lewis acids that each bear at least one di(cyclo)alkylamino group bonded directly thereto, and also benzyltriethylammonium chloride and trisdimethylaminoborane.

Additionally suitable are catalysts from the group of the triaikyibydroxyalkylammonium halides, preferably trialkylhydroxyalkylammonium bromide.

The catalysts are more preferably selected from the group of trialkylhydroxyalkylphosphonium halides, especially preferably trialkylhydroxyalkylphosphonium bromides, most preferably tributylhydroxyethylphosphonium bromide.

The preparation of the tributylhydroxyethylphosphonium bromide catalyst is much less costly compared to the iodine-containing catalyst since the bromoethanol required for the synthesis is available in commercial amounts.

In the process according to the invention, the catalyst is first separated off. It can optionally then be returned to a reaction in unchanged form. The catalyst can also be reused repeatedly. However, it has been observed that there is a fall in reactivity and selectivity after a few cycles.

The process is distinctly improved by the reactivation of the catalyst.

This is done by isolating the catalyst from the reaction mixture and adjusting the halide content to the original stoichiometry by adding a soluble halide salt.

More particularly, the catalyst is reactivated by adding bromide salts selected from the group of ammonium bromide, alkylammonium bromides, alkylphosphonium bromides, hydroxyalkylammonium bromides, hydroxyalkylphosphonium bromides, alkylsulfonium bromides.

The catalyst content in the reaction mixture is between 0.05 and 25 mol %, preferably between 0.5 and 10 mol %, more preferably around 2 mol %.

It has been found that the use of the reactivated catalyst is particularly advantageous for the economic viability of the process. Single-time reuse and also multiple use of the processed catalyst is possible without any significant restriction in reactivity.

It has also been found that, surprisingly, the polarity of the product solution can be lowered by adding a solvent to such a degree that the catalyst salt is absorbed by filtering through a polar stationary phase, and hence the product can be freed continuously from the catalyst.

Suitable solvents for lowering the polarity are especially those from the group of the methyl methacrylates, butyl methacrylates, toluenes, MTBE, alkanes, chlorinated alkanes, preferably hexane, heptane and cyclohexane, and also methylcyclohexane or mixtures thereof.

Stationary phases used are preferably silica gels, kieselguhr, alumina or montmorillonite.

Stabilizers

Suitable stabilizers are known to those skilled in the art. Suitable stabilizers are, for example but not limiting, phenothiazine, tempol, tempo and mixtures thereof.

The applications of cyclic organic, carbonates generally require colorless products. Therefore, for unsaturated compounds, preference is given to non-coloring stabilizers.

Preferably, the stabilizers are selected from the group of substituted phenol derivatives, for example hydroquinone monomethyl ether (HQME), 3,5-di-tert-butyl-4-hydroxytoluene (BHT), 4-methoxyphenol (HQ) and mixtures thereof, optionally in combination with the stabilizers indicated above.

Very particular preference is given to using HQME.

The combination of tempol with HQME is also particularly suitable for the process according to the present invention.

The amount of stabilizer used depends on the starting materials and the nature of the cyclic organic carbonate.

Preference is given to using 20 to 700 ppm, more preferably 100 to 300 ppm, of stabilizer.

No solvents are required for the process according to the invention. As a result, the product attains an optimal space-time yield in the reaction tank.

As a result of the now significantly reduced content of crosslinkers, it is possible to use the product as a resin constituent in formulations of clearcoats, especially since the product via CO₂ insertion has a color number of less than 100. Moreover, there is a rise in the purity of the product as a result of the multitude of side reactions that have been prevented.

Also claimed, therefore, are cyclic organic carbonates prepared according to the process of the present invention, characterized in that the color number of the product is <500, more preferably <100, more preferably <50.

Also claimed are cyclic organic carbonates prepared according to the process of the present invention with a concentration of unsaturated epoxides in the end product of less than 1000 ppm.

Additionally claimed are cyclic organic carbonates prepared according to the process of the present invention with a content of dimethacrylate by-products in the end product of less than 1% by weight.

It has been found that the product is storage-stable since conversion is complete.

The examples which follow are intended to elucidate the invention.

### Example 1: Photometric Determination of the Platinum-Cobalt Color Number in Accordance with DIM ISO 8271

Visual comparison with color standard solutions on the platinum-cobalt scale is replaced by measurement of the absorbance of the sample at the wavelengths of 460 nm and 620 nm. The absorbance differential E₄₆₀ₙₘ−E₆₂₀ₙₘ=ΔE is in a linear relationship with the color purity of the platinum-cobalt standards. When the color number is plotted as a function of ΔE, a calibration line is obtained, the slope of which serves directly as a “factor” for calculation of the color number. A prerequisite is that the sample to be examined corresponds largely to the platinum-cobalt scale in terms of color characteristics, i.e. in terms of hue. Synonyms for the platinum-cobalt color number are APHA (American Public Health Association) or Hazen number.

Procedure

UV/VIS spectrophotometer (for example from Varian, Cary 100), cuvettes of optical specialty glass (path length 50 mm), balance (d=1 mg), standard flasks, volumetric pipettes, 100 ml wide-neck screwtop glass bottle, 10 ml disposable PE pipettes.

Before the actual measurement, it is necessary to examine visually whether the sample corresponds to or differs from the color characteristics (yellow hue, for example by comparison with the standard comparative solutions) of the Pt/Co color scale,

Photometric Measurement

The liquid to be analysed is introduced into a 5 cm cuvette and the cuvette is sealed. It must be free of air bubbles or streaks. Then the absorbance of the sample (front cuvette shaft) is measured with a spectrophotometer at 460 and 620 nm against a cuvette containing demineralized water (back cuvette shaft), and the absorbance differential is calculated. The values b and m can be taken from the calibration curve.

$$\frac{( {Abs}_{460 nm} - {Abs}_{620 nm} ) - b}{m} = \frac{Pt}{Co} colour number$$

$$b = axis intercept , m = slope$$

Since the factors can assume different values in an instrument-specific manner, they should be determined by recording the calibration lines. The factor must be checked annually, if absorbances <0 occur at 620 nm, the difference is likewise formed; in other words, the numerical value of the absorbance at 620 nm is added onto the absorbance at 460 nm. The negative absorbances must not be neglected since they can be manifested in the end result under some circumstances.

### Comparative example 1: Preparation of the tri-n-butyl(2-hydroxyethyl)phosphonium Iodide Catalyst

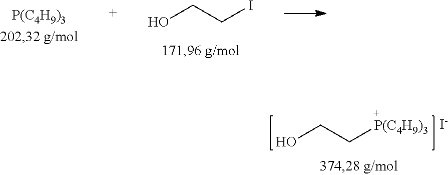

Starting Weights:
267 g (TBP-50EA) (min. 99% tributylphosphine as 50-51% soln. in ethyl acetate) (133.5 g, 0.66 mol) tri-n-butylphosphine (in TBP-50EA from HOKKO Chemicals) (133.5 g, 49-50%) ethyl acetate (in TBP-50EA from HOKKO Chemicals)
180 g (0.686 mol) 2-iodoethanol (99%)

Apparatus:

1 ltr. four-neck round-bottom flask, liquid-phase thermometer, gas inlet tube, sabre stirrer with precision glass stirrer sleeve and stirrer motor, 100 ml dropping funnel, jacketed coil condenser, oil bath with closed-loop temperature control

Procedure:

The tributylphosphine in ethyl acetate was weighed into the nitrogen-purged apparatus. With introduction of nitrogen and stirring, the solution was heated to 60° C. At a liquid-phase temperature of 58° C., the 2-iodoethanol was added dropwise within 61 min (exothermic reaction: in order to avoid a significant temperature rise, the oil bath was removed or lowered somewhat at times): the reaction temperature was kept at ˜60° C.-64° C.). After 24 h at 60° C., the reaction mixture (emulsion) was cooled to RT. The clear yellowish liquid reaction mixture (375.3 g) was concentrated on a rotary evaporator (100° C./5 mbar), giving 254.9 g=103.2% of theory of clear yellowish viscous liquid that crystallized as a white mass in the course of cooling.

Analysis:

³¹P NMR:

94.1 mol % tri-n-butyl(2-hydroxyethyl)phosphonium salt

¹H NMR (secondary phosphorus components were neglected):

97.5/2.5 tri-n-butyl(2-hydroxyethyl)phosphonium salt/2-haloethanol in mol %

### Example 1: Preparation of the tri-n-butyl(2-hydroxyethyl)phosphonium Bromide Catalyst

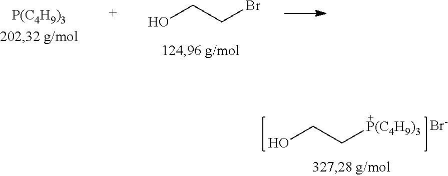

Starting weights:
202.3 g (TBP-50EA) (min. 99% tributylphosphine as 50-51% soln. in ethyl acetate) (101.2 g, 0.50 mol) tri-n-butylphosphine (in TBP-50EA from HOKKO Chemicals) (101.1 g, 49-50%) ethyl acetate (in TBP-50EA from HOKKO Chemicals)
65 g (0.52 mol) 2-bromoethanol (97%)

Apparatus:

500 ml four-neck round-bottom flask, liquid-phase thermometer, gas inlet tube, sabre stirrer with precision glass stirrer sleeve and stirrer motor, 50 ml dropping funnel, jacketed coil condenser, oil bath with closed-loop temperature control

Procedure:

The TBP-50EA (tributylphosphine in ethyl acetate) was initially charged in the nitrogen-purged apparatus owing to its pyrophoric properties. With introduction of nitrogen and stirring, the solution was heated to 60° C. At a liquid-phase temperature of 56° C., the 2-bromoethanol was added dropwise within 40 min (exothermic reaction); the reaction temperature was kept at 60° C. (the oil bath was removed or lowered somewhat at times). After 24 h at 60° C., the reaction mixture (260.0 g of slightly cloudy colorless liquid) was concentrated on a rotary evaporator (100° C./2 mbar), giving 167.6 g (=102.4% of theory) of clear colorless viscous liquid which, after cooling to <30° C., forms a slurry but no homogeneous crystallization.

Analysis:

³¹P NMR:

89.5 mol % tri-n-butyl(2-hydroxyethyl)phosphonium salt

¹H NMR (secondary phosphorus components were neglected):

95.2/4.8 tri-n-butyl(2-hydroxyethyl)phosphonium salt/2-haloethanol in mol %

### Example 2: Preparation of the tri-n-butyl(2-hydroxyethyl)ammonium Bromide Catalyst

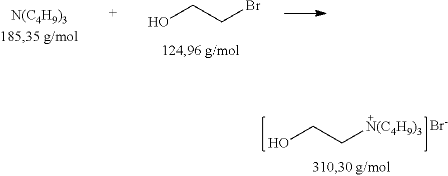

Starting Weights:

139.01 g (0.75 mol) tri-n-butylamine

93.72 g (0.75 mol) 2-bromoethanol (97%)

Apparatus:

500 ml four-neck round-bottom flask, liquid-phase thermometer, sabre stirrer with precision glass stirrer sleeve and stirrer motor, 50 ml dropping funnel, jacketed coil condenser, oil bath with closed-loop temperature control

Procedure:

The tri-n-butylamine was initially charged in the apparatus and heated to ˜80° C. At a liquid-phase temperature of ˜80° C., the 2-bromoethanol was added dropwise within 65 min (non-exothermic reaction): the reaction temperature was kept at SOX. (The reaction mixture is biphasic and is in the form of a cloudy liquid (emulsion) while stirring.) After 24 h at ˜80° C., the reaction mixture was cooled to RT. After the reaction (further reaction at SOX for 24 h), the upper phase (36.4 g, 96% tri-n-butylamine) was removed and the crude product obtained was degassed on a rotary evaporator (90° C./18 mbar), which decreased the mass of the reaction mixture by 4.5 g.

A viscous brown liquid having a purity of 88.5% was obtained.

### Comparative example 2: Reaction with tri-n-butyl(2-hydroxyethyl)phosphonium Iodide (Werner et al., ChemSuSChem, 2014, vol. 7, p. 32SS-3271)

Apparatus:

0.05 ltr. reactor, temperature sensor, stirrer motor with magnetic coupling, oil bath with closed-loop temperature control

Batch:

4.00 g (24.2 mmol) glycidyl methacrylate

208 mg (0,556 mmol, 2 mol % based on epoxide) tri-n-butyl(2-hydroxyethyl)phosphonium iodide

500 ppm (based on epoxide) HQME stabilizer

10 bar CO₂

Procedure:

A 45 ml glass reactor is initially charged with 208 mg (0.556 mmol) of tri-n-butyl(2-hydroxyethyl)phosphonium iodide catalyst and 4.00 g of glycidyl methacrylate (24.2 mmol). The reactor is immersed in an oil hath at 90° C.; purged once with CO₂ and then pressurized (pCO₂=1.0 MPa, 10 bar) and heated for a total of 3 hours. Subsequently, the reactor is cooled to room temperature with an ice bath and the CO₂ is discharged gradually. A sample of the crude product is taken for a GC. The remaining reaction mixture, analogously to the method of Werner et al., is filtered through a silica gel and ail volatile constituents are removed under reduced pressure. The (2-oxo-1,3-dioxolan-4-yl)methyl methacrylate reaction product is obtained as a yellow oil (4.58 g, 23.6 mmol, 98% by NMR).

| | Analysis: | |
|---|---|---|
| | Crude product | After silica gel |
| Pt/Co color number: | >500 (brown in | >500 (brown in |
| | color) | color) |
| GC analysis: | | |
| Glycidyl methacrylate | n.m. (not measured) | n.m. |
| Glycerol carbonate | 0.1 GC area % | n.m. |
| Glycerol trimethacrylate | 0.1 GC area % | 0.1 |
| Glycerol carbonate methacrylate | 97.5 GC area % | 98.1 GC area % |
| Glycerol dimethacrylates | 1.23 GC area % | 0.50 GC area % |
| Glycerol monomethacrylates | 0.36 GC area % | n.m. |

The reaction has excellent reaction times and selectivities on a small scale; the product does not meet the product requirements in the criteria of color number and crosslinker. The previously isolated crude product differed distinctly in this analysis, and so the filtration through silica gel removes not just the catalyst but also polar by-products, such as the hydroxy-functionalized crosslinkers, but on the other hand compounds that are not visible in the GC, such as any silica gel, are incorporated in the reaction mass.

Reaction scale much too small for industrial use—not an example according to the present invention.

### Comparative example 3: Method According to Werner et al., on the Scale of 5 mol of Epoxide, with tri-n-butyl(2-hydroxyethyl)phosphonium Iodide Catalyst

Apparatus:

2.0 ltr. autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

37.4 g (0.10 mol=2 mol % based on epoxide) tri-n-butyl(2-hydroxyethyl)phosphonium iodide

0.356 g (500 ppm based on epoxide) HQME stabilizer

10 bar CO₂

Procedure:

The mixture (without CO₂) was introduced into the autoclave. The autoclave was closed, heated to ˜90° C. while stirring, and then charged with CO₂ to 10 bar (exothermic reaction up to 99° C.). After ˜22 h, the oil bath was switched off/removed and the CO₂ feed was switched off.

Analysis:

| | |
|---|---|
| Pt/Co color number: | >500 (brown in color) |
| GC analysis: | |
| Glycidyl methacrylate | 0.13 GC area % |
| Glycerol carbonate | 1.24 GC area % |
| Glycerol trimethacrylate | 0.21 GC area % |
| Glycerol carbonate methacrylate | 93.5 GC area % |
| Glycerol dimethacrylates | 2.23 GC area % |
| Glycerol monomethacrylates | 0.36 GC area % |

If the experiment described by Werner et al. is scaled up by a factor of 20, there is a significant decline in the purity of the product and an increased level of by-products is formed. The reaction time additionally has to be prolonged at least to 12 h (22 h were used owing to the delayed analysis) in order to obtain a comparable conversion. The color number is still very poor; contamination by 1.24% glycerol carbonate and Σ2.49% crosslinkers. The product does not meet the product requirements; scale-up is not possible without difficulty.

Reaction scale increased, but purity too low, crosslinkers too high, color number too high. Not an example according to the present invention.

### Comparative Example 4: CO₂ Insertion on the Scale of 5 Mol of Epoxide with tri-n-butyl(2-hydroxyethyl)phosphonium Iodide Catalyst, CO₂ Already Added at Room Temperature

Apparatus:

2.0 ltr. autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

37.4 g (0.10 mol=2 mol % based on epoxide) tri-n-butyl(2-hydroxyethyl)phosphonium iodide

0.356 g (500 ppm based on epoxide) HQME stabilizer

10 bar CO₂

Procedure:

The mixture (without CO₂) was introduced into the autoclave. The autoclave was closed, CO₂ was injected to 10 bar and the autoclave was heated up while stirring. At 70° C., the mixture heats up to 90° C. (exothermic reaction): subsequently, the mixture was kept at this temperature by means of an oil bath. After 22 h, the oil bath was switched off/removed and the CO₂ feed was switched off.

Analysis:

| | |
|---|---|
| Pt/Co color number: | >500 (brown in color) |
| GC analysis: | |
| Glycidyl methacrylate | 0.15 GC area % |
| Glycerol carbonate | 0.42 GC area % |
| Glycerol trimethacrylate | n.m. |
| Glycerol carbonate methacrylate | 96.2 GC area % |
| Glycerol dimethacrylates | 0.79 GC area % |
| Glycerol monomethacrylates | 1.14 GC area % |

The scale-up of the experiment by Werner et al. by a factor of 20 can be Improved by adding CO₂ already at room temperature; there is a rise in purity, but the color number is still very poor. Contamination by 0.42 area % of glycerol carbonate and Σ0.79% crosslinkers. The product additionally does not meet the product requirements owing to its color.

Reaction scale increased, crosslinker acceptable, but color number too high and purity moderate. Not an example according to the present invention.

### Comparative Example 5: CO₂ Insertion on the Scale of 5 Mol of Epoxide with tri-n-butyl(2-hydroxyethyl)phosphonium Iodide Catalyst, 5 Bar CO₂ Added at RT

Apparatus:

2.0 ltr. autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

37.4 g (0.10 mol=2 mol % based on epoxide) tri-n-butyl(2-hydroxyethyl)phosphonium iodide

0.356 g (500 ppm based on epoxide) HQME stabilizer

5 bar CO₂

Procedure:

The mixture (without CO₂) was introduced into the autoclave. The autoclave was closed, CO₂ was Injected to 5 bar and the autoclave was heated up to 90° C. while stirring (no significant exothermic reaction), then the mixture was kept at this temperature by means of an oil bath. After 24 h, the oil bath was switched off/removed and the CO₂ feed was switched off.

Analysis:

| | |
|---|---|
| Pt/Co color number: | 371 |
| GC analysis: | |
| Glycidyl methacrylate | 0.32 GC area % |
| Glycerol carbonate | 2.18 GC area % |
| Glycerol trimethacrylate | 1.04 GC area % |
| Glycerol carbonate methacrylate | 91.7 GC area % |
| Glycerol dimethacrylates | 2.69 GC area % |
| Glycerol monomethacrylates | 0.16 GC area % |

5 bar CO₂ with iodide catalysts leads to a distinct decline in conversion and to a very severe deterioration in quality. The catalyst is much too reactive, which means that homogeneous saturation and supply of the reaction solution with CO₂ is no longer possible owing to the lower partial CO₂ pressure.

Reaction scale increased, CO₂ pressure excellent, but crosslinkers, color number and purity unacceptable. Not an example according to the present invention.

### Comparative Example 6: CO₂ Insertion on the Scale of 5 Mol of Epoxide with 4 Mol % of tri-n-butyl(2-hydroxyethyl)phosphonium Iodide Catalyst, 5 Bar CO₂ Added at Room Temperature

Apparatus:

2.0 ltr. autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

74.8 g (0.10 mol=4 mol % based on epoxide) tri-n-butyl(2-hydroxyethyl)phosphonium iodide

0.356 g (500 ppm based on epoxide) HQME stabilizer

5 bar CO₂

Procedure:

The mixture (without CO₂) was introduced into the autoclave. The autoclave was closed, CO₂ was injected to 5 bar and the autoclave was heated up to 90° C. while stirring. At 90° C., the mixture heats up to ˜95° C. (slightly exothermic reaction); subsequently, the mixture was kept at SOX by means of an oil bath. After ˜24 h, the oil bath was switched off/removed and the CO₂ feed was switched off.

Analysis:

| | |
|---|---|
| Pt/Co color number: | 437 |
| GC analysis: | |
| Glycidyl methacrylate | 4.12 GC area % |
| Glycerol carbonate | 2.34 GC area % |
| Glycerol trimethacrylate | 0.43 GC area % |
| Glycerol carbonate methacrylate | 88.8 GC area % |
| Glycerol dimethacrylates | 4.22 GC area % |
| Glycerol monomethacrylates | 0.82 GC area % |

An increase in the catalyst charge confirms the effect of a CO₂ undersupply. The side reactions that arise in the absence of CO₂ very significantly worsen the product quality, and there is additionally a massive decline in conversion.

Reaction scale increased, CO₂ pressure excellent, but crosslinkers, color number and purity unacceptable. Not an example according to the present invention.

### Comparative Example 7: CO₂ Insertion on the Scale of 5 Mol of Epoxide with 2 Mol % of tri-n-butyl(2-hydroxyethyl)phosphonium Bromide Catalyst, 10 Bar CO₂ Added at Room Temperature

Apparatus:

2.0 ltr. autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.356 g (500 ppm based on epoxide) HQME stabilizer

10 bar CO₂

Procedure:

The mixture (without CO₂) was introduced into the autoclave. The autoclave was closed, CO₂ was injected to 10 bar and the autoclave was heated up to 90° C. while stirring. Above 80° C., the mixture heats up to ˜106° C. (strongly exothermic, reaction); subsequently, the mixture was kept at 90° C. by means of an oil bath. After ˜24 h, the oil bath was switched off/removed and the CO₂ feed was switched off.

Analysis:

| | |
|---|---|
| Pt/Co color number: | 36 |
| GC analysis: | |
| Glycidyl methacrylate | 0.31 GC area % |
| Glycerol carbonate | 1.47 GC area % |
| Glycerol trimethacrylate | 0.13 GC area % |
| Glycerol carbonate methacrylate | 91.9 GC area % |
| Glycerol dimethecrylates | 2.80 GC area % |
| Glycerol monomethacrylates | 1.31 GC area % |

The reaction of the bromide catalyst, as described in the literature, at first glance is initially less selective and slightly less active, in strong contrast to this, the reaction, however, is much more exothermic. Since the reaction is formally the same reaction except that the catalyst has been changed, the bromide catalyst does not seem to lower the activation energy to the extent enabled by the iodide catalyst since the same heat of reaction is released abruptly. This has the advantage that the reaction is less marked at room temperature, but the disadvantage that, on attainment of the necessary activation energy, a very much larger amount of reactants is present (since no reaction has taken place yet), as a result of which a large amount of energy is released in a short time. The consequence is the observed overheating of the reactor system to 106° C. Moreover, there is a very remarkable and distinct decline in the color number; the iodide catalyst has a distinct adverse effect on the color number of the product.

Reaction scale increased, bromide catalyst used, color number very good, but crosslinkers and purity unacceptable. Not an example according to the present invention.

### Comparative Example 8: CO₂ Insertion on the Scale of 5 Mol of Epoxide with 2 Mol % of tri-n-butyl(2-hydroxyethyl)phosphonium Bromide Catalyst, 10 Bar CO₂ Added at RT

Note: Owing to the high exothermicity in Comparative Example 7, there was a considerable polymerization hazard in further experiments. For this reason, a glass inlay was used in the autoclave hereinafter, which would enable opening of the autoclave on polymerization of the mixture and would prevent the total loss thereof. At the same time, it was possible in this way to safely test lesser stabilization, even though this additionally increased the risk of polymerization. The change from Comparative Example 7 is thus in apparatus and stabilizer content.

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.356 g (150 ppm based on epoxide) HQME stabilizer

10 bar CO₂

Procedure:

The mixture was weighed into the flat-bottomed glass vessel. The mixture in the flat-bottomed glass vessel was brought into solution with a glass rod, forming a colorless solution. The flat-bottomed glass vessel containing the mixture (without CO₂) was inserted into the autoclave. The autoclave was closed, CO₂ was injected to 10 bar and the autoclave was heated up to 90° C. while stirring. Above 90° C., the mixture heats up to ˜113° C. (strongly exothermic reaction, poorer removal of heat through glass inlay); subsequently, the mixture was kept at 90° C. by means of an oil bath. After 24 h, the oil bath was switched off/removed and the CO₂ feed was switched off.

Analysis:

| | |
|---|---|
| Pt/Co color number: | 39 |
| GC analysis: | |
| Glycidyl methacrylate | 0.04 GC area % |
| Glycerol carbonate | 2.12 GC area % |
| Glycerol trimethacrylate | 0.57 GC area % |
| Glycerol carbonate methacrylate | 91.2 GC area % |
| Glycerol dimethacrylates | 3.25 GC area % |
| Glycerol monomethacrylates | 0.54 GC area % |

The overheating of the reaction has risen once again by 7° C. as a result of use of a glass inlay for the autoclave, which Is entirely plausible by virtue of the now poorer removal of heat, in spite of lower stabilization, the batch has not polymerized in spite of a strongly exothermic reaction; stabilization with 150 ppm of HQME is thus sufficiently high even for unexpected events. The final product can also be stabilized with even less HQME. The additional temperature peak of 7° C. increases the formation of the crosslinker content perceptibly once again. However, the resultant exothermicity has to be removed urgently and overheating has to be prevented.

Reaction scale increased, bromide catalyst used, color number very good, but crosslinkers and purity unacceptable. Temperature too high. Not an example according to the present invention.

### Comparative Example 9: CO₂ Insertion on the Scale of 5 Mol of Epoxide with 2 Mol % of tri-n-butyl(2-hydroxyethyl)phosphonium Bromide Catalyst, 5 Bar CO₂ Added at Room Temperature

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.356 g (150 ppm based on epoxide) HQME stabilizer

5 bar CO₂

Procedure:

The mixture was weighed into the flat-bottomed glass vessel. The mixture in the flat-bottomed glass vessel was brought into solution with a glass rod, forming a colorless solution. The flat-bottomed glass vessel containing the mixture (without CO₂) was inserted into the autoclave. The autoclave was closed, CO₂ was injected to 5 bar and the autoclave was heated up to 90° C. while stirring. After 20 min at 90° C., the mixture heats up to ˜98° C. (exothermic reaction, poor removal of heat through glass inlay); subsequently, the mixture was kept at 90° C. by means of the oil bath. After ˜24 h, the oil bath was switched off/removed and the CO₂ feed was switched off.

Analysis:

| | |
|---|---|
| Pt/Co color number: | 38 |
| GC analysis: | |
| Glycidyl methacrylate | 0.04 GC area % |
| Glycerol carbonate | 2.08 GC area % |
| Glycerol trimethacrylate | 0.62 GC area % |
| Glycerol carbonate methacrylate | 91.6 GC area % |
| Glycerol dimethacrylates | 3.02 GC area % |
| Glycerol monomethacrylates | 0.36 GC area % |

Completely surprisingly, with the bromide catalyst, the halved partial CO₂ pressure does not have any apparent adverse effect in the experimental result—this having caused a distinct deterioration with the iodide catalyst. By contrast, the product quality rises marginally as a result of a fall in crosslinker content. Color number is unchanged within the scope of measurement accuracy. Reaction scale increased, bromide catalyst used and optimized CO₂ pressure, color number very good, but crosslinkers and purity unacceptable. Temperature too high. Not an example according to the present invention.

### Example 3: Determination of the Breakdown Temperature of Glycerol Carbonate Methacrylate

A sample of glycerol carbonate methacrylate was examined for its loss of mass by means of thermogravimetric analysis, firstly in the range from room temperature to 500° C. at heating rate of 5 K/min, see FIG. 1.

A distinct loss of mass already begins just below 100° C. Since this is considerably higher at the boiling point at standard pressure, it can be assumed that a breakdown reaction of the glycerol carbonate is already commencing at 100° C.

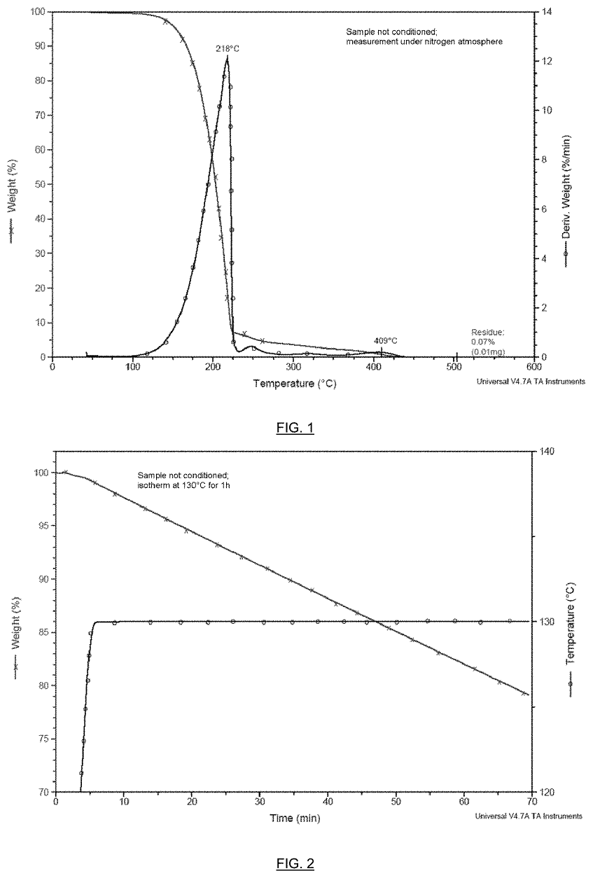

In a second thermogravimetric analysis, a sample of glycerol carbonate methacrylate was stored isothermally in each case at 60° C. for 16 h, 100° C. for 4 h and 130° C. for 1 h, see FIG. 2.

Storage at 130° C. causes a loss of mass of more than 20% by weight within 60 min. The product is unstable at 130° C.

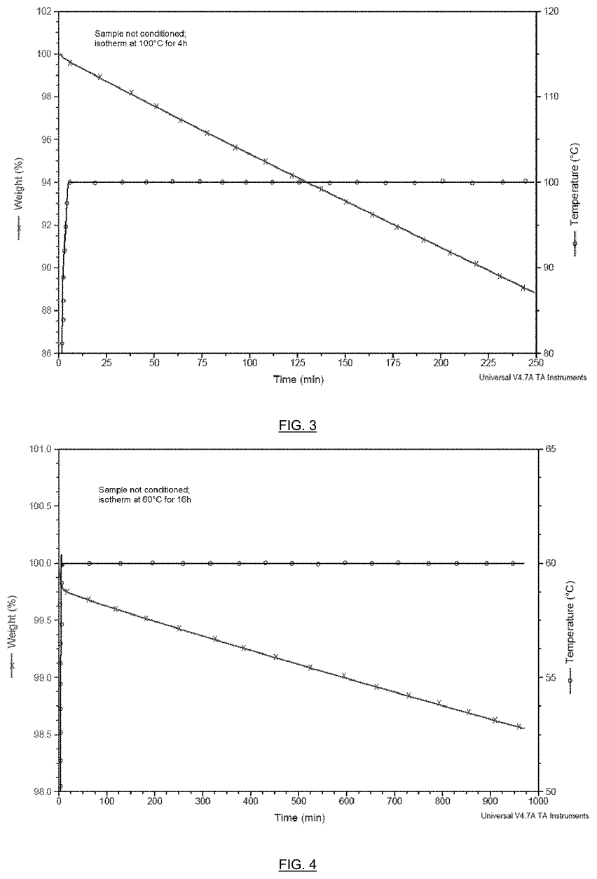

Storage at 100° C. causes a loss of mass of 12% by weight within 240 min. The product is unstable at 100° C., see FIG. 3.

Storage at 60° C. causes a loss of mass of ˜1% by weight within 1000 min, see FIG. 4. The product is stable at this temperature; the breakdown temperature is in the range of 60-100° C., probably just below 90° C., since the initial loss of mass in the TGA analysis commences here at a heating rate of 5 K/min. The synthesis of glycerol carbonate methacrylate should therefore be limited to a temperature below 90° C.

### Comparative Example 10: CO₂ Insertion on the Scale of 5 Mol of Epoxide with 2 Mol % of tri-n-butyl(2-hydroxyethyl)phosphonium Bromide Catalyst, 5 Bar CO₂ Added at Room Temperature, Exothermicity Limit

Apparatus:

2.0 ltr. autoclave, flat-bottomed glass vessel as Insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.356 g (150 ppm based on epoxide) HQME stabilizer

5 bar CO₂

Procedure:

The mixture was weighed into the flat-bottomed glass vessel. The mixture in the flat-bottomed glass vessel was brought into solution with a glass rod, forming a colorless solution. The flat-bottomed glass vessel containing the mixture (without CO₂) was inserted into the autoclave. The autoclave was closed, CO₂ was injected to 5 bar and the autoclave was heated up to 70° C. while stirring. After 5 min at 70° C., the mixture heats up beyond the oil bath temperature. The temperature is 81° C. after 5 min; after 8 min at 83° C., the oil bath is removed; after a total of 20 min, the maximum temperature of ˜91° C. has been attained, which is maintained in spite of air cooling for 30 min, and so the mixture was cooled back down to 65° C. with a water bath within 15 min. The mixture was kept at 70° C. by means of the oil bath. After a total reaction time of ˜24 h, the oil bath was switched off/removed and the CO₂ feed was switched off.

Analysis:

| | |
|---|---|
| Pt/Co color number: | 36 |
| GC analysis: | |
| Glycidyl methacrylate | 2.25 GC area % |
| Glycerol carbonate | 1.56 GC area % |
| Glycerol trimethacrylate | 0.19 GC area % |
| Glycerol carbonate methacrylate | 90.0 GC area % |
| Glycerol dimethacrylates | 2.6 GC area % |
| Glycerol monomethacrylates | n.m. |

By comparison with a reaction temperature around 90° C., the reaction becomes much more selective when the maximum temperature is limited. As expected, the conversion falls as a result of the now lower reaction temperature, there is a distinct decline in the crosslinker content and no hydrolysis product is formed any longer. Since it is now sufficiently well known that the side reaction occurs when the reaction proceeds quickly and, as a result, too little CO₂ is present, the reaction will from now on be quenched at even lower temperature (75° C.) by cooling, but on the other hand operated at higher temperature for further reaction if it is sufficiently slow to restore the CO₂ saturation. If the CO₂ saturation is sufficiently high, it would also be necessary to be able to suppress the decomposition reaction above 90° C.

Reaction scale increased, bromide catalyst used and optimized CO₂ pressure, color number very good, but crosslinkers and purity not good enough. Not an example according to the present invention.

### Comparative Example 9: CO₂ Insertion on the Scale of 5 Mol of Epoxide with 2 Mol % of tri-n-butyl(2-hydroxyethyl)phosphonium Bromide Catalyst, 5 Bar CO₂ Added at Room Temperature, Exothermicity Limit of 85° C., Further Reaction at 90° C.

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.356 g (150 ppm based on epoxide) HQME stabilizer

5 bar CO₂

Procedure:

The mixture was weighed into the flat-bottomed glass vessel. The mixture in the flat-bottomed glass vessel was brought into solution with a glass rod, forming a colorless solution. The flat-bottomed glass vessel containing the mixture (without CO₂) was inserted into the autoclave. The autoclave was closed, CO₂ was Injected to 5 bar and the autoclave was heated up to 70° C. while stirring. Shortly before internal temperature 70° C., the oil bath was removed. The mixture heats up further of its own accord. After 25 min, the temperature is 85° C., and so it was briefly (15 min) cooled back down to 77° C. with a water bath. The mixture was then trace-heated by the oil bath at 70° C., as a result of which another temperature peak up to 82° C. was observed. After abatement thereof, the oil bath temperature was increased to 90° C. and the further reaction phase was commenced. After a total reaction time of ˜24 h, the oil bath was switched off/removed and the CO₂ feed was switched off.

Analysis:

| | After 30 min | After 24 h (product) |
|---|---|---|
| Pt/Co color number: | n. d. | 25 |
| | HPLC analysis | GC analysis: |
| Glycidyl methacrylate | 91.6 w % | 1.18 GC area % |
| Glycerol carbonate | not visible | 1.16 GC area % |
| Glycerol trimethacrylate | n.m. | n.m. |
| Glycerol carbonate methacrylate | 4.79 w % | 91.9 GC area % |
| Glycerol dimethacrylates | 1.54 w % | 2.09 GC area % |
| Glycerol monomethacrylates | 1.58* yr % | n.m. |

*in HPLC an unreliable value as a result of the glycidyl hydrolysis

The temperature limit is exceedingly beneficial to product quality and measurably improves the color number, eliminates the triple crosslinker, increases the product purity, and, as a result of the higher further reaction temperature, a higher conversion is also achieved, in spite of otherwise excellent improvement, the crosslinker content is still outside the product specification. However, sampling of the reaction after 30 min showed that virtually the entire crosslinker content had already been formed at this time. The catalyst is already active at RT, but much less marked than the iodide catalyst. For this reason, the CO₂ should be In contact with the reaction solution upstream of the catalyst.

Reaction scale increased, bromide catalyst used and optimized CO₂ pressure, color number very good, but crosslinkers and purity still not good. Not an example according to the present invention.

### Example 4: Phosphonium Bromide Catalyst has Contact with the Reaction Solution Only after CO₂ as a Result of Prior Dry Ice Addition

Note: It would have been desirable to add the catalyst only after the reactor had reached 5 bar CO₂.

Owing to the viscosity, the low catalyst weight and the dead volume of the riser tube in the autoclave, this has not been possible on a small scale, but will be essential for larger scales. See Example 10: Scale-up of Example 8 to a 22.5 mol (6 l) batch and others on a scale greater than 20 mol.

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)phosphonium bromide

0.107 g (150 ppm based on epoxide) HQME stabilizer

5 bar CO₂

Procedure:

The mixture was weighed into the flat-bottomed glass vessel, but, before the amount of catalyst was added, about 6 g of dry ice were Introduced info the flat-bottomed glass vessel, and there was no homogenization with the glass rod. The flat-bottomed glass vessel containing the mixture was inserted immediately into the autoclave. The autoclave was closed and the stirring was switched on. The autoclave was permanently charged with CDs to 5 bar in order to replace reacting CO₂. The autoclave was heated up stepwise to 70° C. (+10° C. every 15 min). On attainment of 70° C., the enthalpy of reaction of the mixture is sufficient to heat it to 85° C. without further heating, and so counter-cooling with a water bath was effected if required to limit the temperature to 75° C. For further reaction after reaction time 5 h (25% by weight of epoxide present in solution), the autoclave was heated to 85° C. After 24 h, the oil bath was removed, the reaction was cooled down and the CO₂ feed was switched off.

Analysis:

| | |
|---|---|
| Pt/Co color number: | 17 |
| GC analysis: | |
| Glycidyl methacrylate | 0.31 GC area % |
| Glycerol carbonate | n.m. |
| Glycerol trimethacrylate | n.m. |
| Glycerol carbonate methacrylate | 96.6 GC area % |
| Glycerol dimethacrylates | 0.51 GC area % |
| Glycerol monomethacrylates | n.m. |

Color number very good, no contamination by glycerol carbonate and crosslinker with 0.51 GC area % within the specification range. The product meets the product requirements. The further reaction temperature must accordingly be below 90° C., However, this may be a product-specific parameter and would have to be examined for other products by means of TGA in each case with regard to the breakdown temperature, if the reaction conditions do not provide adequate product quality. The sequence such that the catalyst may not be added to the solution until after contact with CO₂ is absolutely crucial.

Reaction scale increased, bromide catalyst used and optimized CO₂ pressure, color number, crosslinkers and purity very good. Example according to the present invention.

### Example 5: Alkylammonium Bromide Catalyst

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

32.7 g (0.10 mol=2 mol % based on epoxide) tri-n-butyl(2-hydroxyethyl)ammonium bromide

0.107 g (150 ppm based on epoxide) HQME stabilizer

5 bar CO₂

Procedure:

The mixture was weighed into the flat-bottomed glass vessel, but, before the amount of catalyst was added, about 6 g of dry ice were introduced into the flat-bottomed glass vessel, and there was no homogenization with the glass rod. The flat-bottomed glass vessel containing the mixture was inserted immediately into the autoclave. The autoclave was closed and the stirring was switched on. The autoclave was permanently charged with CO₂ to 5 bar in order to replace reacting CO₂. The autoclave was heated up stepwise to 70° C. (+10° C. every 15 min). On attainment of 70° C., the enthalpy of reaction of the mixture is sufficient to heat it to 85° C. without further heating, and so counter-cooling with a water bath was effected if required to limit the temperature to 75° C. For further reaction after reaction time 5 h (25% by weight of epoxide remaining in solution), the autoclave was left at 85° C. After ˜24 h, the oil bath was removed, the reaction was cooled down and the CO₂ feed was switched off.

Analysis:

| | |
|---|---|
| Pt/Co color number: | 61 |
| GC analysis: | |
| Glycidyl methacrylate | 1.28 GC area % |
| Glycerol carbonate | 0.17 GC area % |
| Glycerol trimethacrylate | n.m. |
| Glycerol carbonate methacrylate | 94.3 GC area % |
| Glycerol dimethacrylates | 0.22 GC area % |
| Glycerol monomethacrylates | 0.3 GC area % |

The color number is not very good, slight contamination by 0.17 GC area % of glycerol carbonate and crosslinkers at 0.22 GC area %, even lower than before and also within the specification range. Owing to the high epoxide content, the product does not meet the product demands, but the conversion can be increased at the expense of production costs by longer reaction time. The catalyst Is indeed suitable, hut slightly slower. The new sequence of addition now makes catalyst systems other than phosphorus salts possible.

Reaction scale increased, catalyst system extended with optimized CO₂ pressure, color number, crosslinkers and purity very good. Example according to the present invention.

### Example 6: Tricyclohexyl(2-hydroxyethyl)phosphonium Bromide Catalyst has Contact with the Reaction Solution Only after CO₂ as a Result of Prior Dry Ice Addition

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

40.5 g (0.10 mol=2 mol % based on epoxide) tricyclohexyl(2-hydroxyethyl)phosphonium bromide (preparation analogous to Example 1: Preparation of the tri-n-butyl(2-hydroxyethyl)phosphonium bromide catalyst)

0.107 g (150 ppm based on epoxide) HQME stabilizer

5 bar CO₂

Procedure:

The mixture was weighed into the flat-bottomed glass vessel, but, before the amount of catalyst was added, about 6 g of dry ice were introduced into the flat-bottomed glass vessel, and there was no homogenization with the glass rod. The flat-bottomed glass vessel containing the mixture was inserted immediately into the autoclave. The autoclave was closed and the stirring was switched on. The autoclave was permanently charged with CO₂ to 5 bar in order to replace reacting CO₂. The autoclave was heated up stepwise to 70° C. (+10° C. every 15 min). On attainment of 70° C., the enthalpy of reaction of the mixture is sufficient to heat it to 85° C. without further heating, and so counter-cooling with a wafer bath was effected if required to limit the temperature to 75° C. For further reaction after reaction time 5 h (˜25% by weight of epoxide remaining in solution), the autoclave was left at 70° C., After ˜24 h, the oil bath was removed, the reaction was cooled down and the CO₂ feed was switched off.

Analysis:

| | |
|---|---|
| Pt/Co color number: | 23 |
| GC analysis: | |
| Glycidyl methacrylate | 0.291 GC area % |
| Glycerol carbonate | n.m. |
| Glycerol trimethacrylate | n.m. |
| Glycerol carbonate methacrylate | 96.5 GC area % |
| Glycerol dimethacrylates | 0.52 GC area % |
| Glycerol monomethacrylates | n.m. |

No relevant difference from Example 4 according to the present invention.

Reaction scale increased, ligands for the catalyst system extended with optimized CO₂ pressure, color number, crosslinkers and purity very good. Example according to the present invention.

### Example 7: Tri-n-octyl(2-hydroxyethyl)phosphonium Bromide Catalyst has Contact with the Reaction Solution Only after CO₂ as a Result of Prior Dry Ice Addition

Apparatus:

2.0 ltr. autoclave, flat-bottomed glass vessel as Insert for the autoclave, temperature sensor, stirrer motor, oil bath with closed-loop temperature control, riser tube for sampling, fittings (<60 bar, non-return valve), balance

Batch:

710.8 g (5.00 mol) glycidyl methacrylate

49.56 g (0.10 mol=2 mol % based on epoxide) tri-n-octyl(2-hydroxyethyl)phosphonium bromide (preparation analogous to Example 1: Preparation of the tri-n-butyl(2-hydroxyethyl)phosphonium bromide catalyst)

0.107 g (150 ppm based on epoxide) HQME stabilizer

5 bar CO₂

Procedure:

The mixture was weighed into the flat-bottomed glass vessel, but, before the amount of catalyst was added, about 6 g of dry ice were introduced into the flat-bottomed glass vessel, and there was no homogenization with the glass rod. The flat-bottomed glass vessel containing the mixture was inserted immediately into the autoclave. The autoclave was closed and the stirring was switched on. The autoclave was permanently charged with CO₂ to 5 bar in order to replace reacting CO₂. The autoclave was heated up stepwise to 70° C. (+10° C. every 15 min). On attainment of 70° C., the enthalpy of reaction of the mixture is sufficient to heat it to 85° C. without further heating, and so counter-cooling with a water bath was effected if required to limit the temperature to 75° C. For further reaction after reaction time 5 b (25% by weight of epoxide remaining in solution), the autoclave was left at 70° C. After 24 h, the oil bath was removed, the reaction was cooled down and the CO₂ feed was switched off.

Analysis:

| | |
|---|---|
| Pt/Co color number: | 24 |
| GC analysis: | |
| Glycidyl methaciylate | 0.28 GC area % |
| Glycerol carbonate | n.m. |
| Glycerol trimethacrylate | n.m. |
| Glycerol carbonate methacrylate | 96.4 GC area % |
| Glycerol dimethacrylates | 0.49 GC area % |
| Glycerol monomethacrylates | n.m. |

No relevant difference from Example 4 according to the present invention.

Reaction scale increased, ligands for the catalyst system extended once again with optimized CO₂ pressure, color number, crosslinkers and purity very good. Example according to the present invention.

### Example 8: Transferring tri-n-butyl(2-hydroxyethyl)phosphonium Bromide Catalyst in Acetonitrile into the Autoclave Via HPLC Pump at CO₂ Pressure 5 Bar

Apparatus:

2.0 ltr. autoclave, flat-bottomed glass vessel as insert for the autoclave, temperature sensor, stirrer motor, oil bath with closed-loop temperature control, riser tube for sampling, fittings (<60 bar, non-return valve), balance, HPLC pump (Knauer Smartline 100 HPLC pump with 50 ml TI pump head)

Batch:

710.8 g (5.00 mol) glycidyl methacrylate

32.7 g (0.10 mol=2 mol % based on epoxide) tri-n-butyl(2-hydroxyethyl)phosphonium bromide

0.107 g (150 ppm based on epoxide) HQME stabilizer

5 bar CO₂

Procedure:

The mixture was weighed into the flat-bottomed glass vessel. The flat-bottomed glass vessel containing the mixture was inserted immediately into the autoclave. The autoclave was closed and the stirring was switched on. The autoclave was charged with CO₂ to 5 bar and opened up to the CO₂ reservoir in order to replace reacting CO₂. The catalyst was dissolved in acetonitrile and transferred with an HPLC pump via the riser tube for sampling into the autoclave pressurized to 5 bar. The pump and conduit were purged with 1.5 eq. of the dead volume of acetonitrile. The autoclave was heated up stepwise to 70° C. (+10° C. every 15 min). On attainment of 70° C., the enthalpy of reaction of the mixture is sufficient to heat it to 85° C. without further heating, and so counter-cooling with a water bath was effected if required to limit the temperature to 75° C. For further reaction after reaction time 5 h (25% by weight of epoxide remaining in solution), the autoclave was left at 70° C. After 31 h, the oil bath was removed, the reaction was cooled down and the CO₂ feed was switched off.

Analysis:

| | |
|---|---|
| Pt/Co color number: | 17 |
| GC analysis: | |
| Glycidyl methacrylate | 0.01 GC area % |
| Glycerol carbonate | n.m. |
| Glycerol trimethacrylate | n.m. |
| Glycerol carbonate methacrylate | 95.6 GC area % |
| Glycerol dimethacrylates | 0.43 GC area % |
| Glycerol monomethacrylates | n.m. |

No relevant difference from Example 4 according to the present invention. The extension in the further reaction time leads to a product with 100 ppm of glycidyl methacrylate, as a result of which there is no longer any labelling obligation.

### Example 9: Continuous Removal of the Catalyst (tri-n-butyl(2-hydroxyethyl)phosphonium Bromide)

For separation of the catalyst from the product, the polarity of the mixture was first adjusted such that the catalyst is not eluted on contact with silica gel. Different nonpolar solvents were tested, and preference was given to those that had unlimited miscibility with glycerol carbonate methacrylate.

For evaluation as to whether a product/solvent mixture is sufficiently nonpolar, the catalyst (as a solution in acetonitrile) was applied to a silica gel-coated thin-layer chromatography card (aluminium TIC foils 5×7.5 cm, silica gel 60 F 254), the position was marked with a pencil and then the chromatograph was developed in the solvent to be tested. The maximum solvent front was marked and the dried TLC card was briefly painted with a 10% aqueous silver nitrate solution. The card was left to dry again and then developed under UV light at 254 and 365 nm for 10 seconds.

The catalyst or silver halide formed is thus visible as a brown spot. In the case of a suitable solvent, the catalyst has not moved from the starling mark, which is the case in the case of glycerol carbonate methacrylate particularly for toluene, MTBE and dichloromethane.

To adjust the polarity, the product was purified by chromatography with dichloromethane using silica gel. A catalyst-free glycerol carbonate methacrylate thus obtained was used to create a polarity series (1:1 to 1:10 (product to solvent in parts by volume)) by diluting with solvent (toluene, MTBE, dichloromethane, etc.). The catalyst was again applied (as a solution in acetonitrile) to a silica gel-coated thin-layer chromatography card (aluminium TLC foils 5×7.5 cm, silica gel 60 F 254) and the position was marked with a pencil. However, this TLC card was developed in the polarity series ascertained above in each case, it was thus possible to determine the concentration for each solvent in which the product as a mixture with the solvent itself was nonpolar enough not to elute the catalyst itself from the stationary silica gel phase.

For glycerol carbonate methacrylate, the minimum mixing ratio thus ascertained is 1 part by volume carbonate to 2 parts by volume toluene, and so a 33.3% by volume solution of glycerol carbonate methacrylate in toluene is obtained.

Apparatus:

160.0 g of silica gel (silica gel 60 [0.035 to 0.07 mm]) [dry (as supplied)]

HPLC pump (KNAUER Smartline 100 HPLC pump with 50 ml pump head made from titanium) for the conveying of the product solution or MeOH (purge soln.), pressure relief valve (opening pressure: 24 bar (Swagelok pressure relief valve, nominal opening pressure: 3.5 to 24 bar), manometer (0-100 bar) to display the column supply pressure, glass chromatography column (Gotec Labortechnik GmbH, designation: “SC” 600-26, Article No: G.20253, column volume: 283-326 ml, max. pressure: 50 bar, inlet with filter frit and outlet with filter frit and filter [type: F, >25 μm=>0.025 mm]), thermostat (to control the temperature of the glass chromatography column), 16-fold valve with drive (Knauer SmartLine AWA 30 BK for time-controlled sampling), balances (to ascertain the decrease in mass of the feed)

Procedure:

Column temperature control: none (RT); flow rate: 10 ml/min; residence time: 21.85 min (Volume of the column [305 ml] minus filling [160 g/1.85 g/ml=86.5 ml] divided by flow rate [10.0 ml/min])

The silica gel introduced into MeOH was transferred into the glass column (fill height of the packing: 120 mm=12 mi). The column packing was flushed at RT with ˜670 ml (3 times the usable volume)=530 g of MeOH (10 ml/min). The column packing was made ready by flushing at RT with ˜610 ml (2.8 times the usable volume)=530 g of toluene (10 ml/min). The product solution (˜740 ml, 33.3% by volume solution of glycerol carbonate methacrylate in toluene) was applied to the column packing at RT at 10 mi/minute and the eluates obtained were collected in a 4-minute cycle. The column packing was flushed at RT with 610 ml (2.8 times the usable volume)=530 g of toluene (10 ml/min), and the eluates were collected in a 4-minute cycle. Thereafter, the catalyst was flushed out with ˜720 ml (3.3 times the usable volume)=570 g of MeOH (10 ml/min), and the eluates were collected in a 4-minute cycle.

| | | | Pump | | Starting | | |
|---|---|---|---|---|---|---|---|
| Time | Delivery rate | | pressure | | weight | | Comment |
| 8:03 | 0 | g/min | 4.6 | bar | 0 | | Commencement of column flush (10 ml/min |
| | | | | | | | MeOH) commenced, silica gel filling adjusted |
| | | | | | | | by ~15 mm to ~575 mm, for reduction of dead |
| | | | | | | | volume the feed was screwed downward |
| 08:10 | 7.9 | g/min | 13 | bar | 52.1 | g | |
| 08:41 | 7.7 | g/min | 12.6 | bar | 295.3 | g | Column supply pressure: 0 bar |
| | | | | | | | MeOH flush ended, HPLC pump pre-flushed with |
| 09:11 | 7.8 | g/min | 13.3 | bar | 530.7 | g | toluene |
| 09:14 | 211.4 | g/min | 13.3 | bar | 0.5 | g | Commencement of column flush commenced at |
| | | | | | | | 10 ml/min of toluene |
| 09:43 | 8.6 | g/min | 12.6 | bar | 249.9 | g | Zero column supply pressure |
| 10:16 | 8.7 | g/min | 13 | bar | 533.9 | g | Sampling valve flushed |
| | | | | | | | Feeding of the product/toluene solution - toluene |
| 10:25 | 1.1 | g/min | 12.8 | bar | 0.7 | g | [1:2] commenced at 10 ml/min, commencement of |
| | | | | | | | sampling 10-A.1 |
| 10:30 | 10.3 | g/min | 13.6 | bar | 54.2 | g | Commencement of sampling 10-A.2 |
| 10:32 | 10 | g/min | 13.5 | bar | 74.5 | g | Zero column supply pressure |
| 10:34 | 9.9 | g/min | 13.8 | bar | 94.4 | g | Commencement of sampling 10-A.3 |
| 10:38 | 10.1 | g/min | 13.6 | bar | 134.3 | g | Commencement of sampling 10-A.4 |
| 10:42 | 9.9 | g/min | 14.1 | bar | 174.3 | g | Commencement of sampling 10-A.5 |
| 10:46 | 9.9 | g/min | 14.3 | bar | 214 | g | Commencement of sampling 10-A.6 |
| 10:50 | 9.9 | g/min | 14.5 | bar | 253.5 | g | Commencement of sampling 10-A.7 |
| 10:54 | 9.9 | g/min | 14.5 | | 292.9 | | Commencement of sampling 10-A.8 |
| 10:58 | 9.9 | g/min | 14.5 | bar | 332.4 | g | Commencement of sampling 10-A.9 |
| 11:01 | 9.6 | g/min | 14.8 | bar | 357.7 | g | Zero column supply pressure |
| 11:02 | 9.9 | g/min | 15 | bar | 367.6 | g | Commencement of sampling 10-A.10 |
| 11:06 | 9.9 | g/min | 15.1 | bar | 407.4 | g | Commencement of sampling 10-A.11 |
| 11:10 | 9.9 | g/min | 15.1 | bar | 447.1 | g | Commencement of sampling 10-A.12 |
| 11:14 | 9.9 | g/min | 15.8 | bar | 486.7 | g | Commencement of sampling 10-A.13 |
| 11:18 | 9.9 | g/min | 15.3 | bar | 526.2 | g | Commencement of sampling 10-A.14 |
| 11:22 | 10 | g/min | 15.1 | | 565.8 | | Commencement of sampling 10-A.15 |
| 11:26 | 9.9 | g/min | 15.8 | bar | 605.3 | g | Commencement of sampling 10-A.16 |
| 11:30 | 9.8 | g/min | 15.8 | bar | 644.7 | g | Commencement of sampling 10-A.17 |
| 11:34 | 9.7 | g/min | 15.8 | bar | 684 | g | Commencement of sampling 10-A.18 |
| 11:35 | | g/min | 8.3 | bar | | | Flow briefly stopped (soln. transferred) |
| 11:38 | 9.9 | g/min | 15.8 | | 26.6 | | Commencement of sampling 10-A.19 |
| 11:40 | 6.2 | g/min | 8.6 | bar | 42.7 | g | Change to toluene (10 ml/min) |
| 11:42 | 8.7 | g/min | 14 | bar | 14.2 | g | Commencement of sampling 10-B.1 |
| 11:46 | 8.6 | g/min | 14.1 | bar | 48.6 | g | Commencement of sampling 10-B.2 |
| 11:50 | 8.6 | g/min | 13.8 | bar | 83 | g | Commencement of sampling 10-B.3 |
| 11:54 | 8.6 | g/min | 13.6 | bar | 117.4 | g | Commencement of sampling 10-B.4 |
| 11:58 | 8.6 | g/min | 13.5 | | 151.9 | | Commencement of sampling 10-B.5 |
| 12:02 | 8.6 | g/min | 13.5 | bar | 186.3 | g | Commencement of sampling 10-B.6 |
| 12:06 | 8.6 | g/min | 13.3 | bar | 220.7 | g | Commencement of sampling 10-B.7 |
| 12:10 | 8.5 | g/min | 13.3 | bar | 255 | g | Commencement of sampling 10-B.8 |
| 12:14 | 8.7 | g/min | 13.6 | bar | 289.5 | g | Commencement of sampling 10-B.9 |
| 12:18 | 8.6 | g/min | 13.1 | bar | 323.8 | g | Commencement of sampling 10-B.10 |
| 12:22 | 8.6 | g/min | 13.3 | bar | 358.2 | g | Commencement of sampling 10-B.11 |
| 12:26 | 8.6 | g/min | 13.5 | bar | 392.6 | g | Commencement of sampling 10-B.12 |
| 12:30 | 8.5 | g/min | 13.3 | | 426.9 | | Commencement of sampling 10-B.13 |
| 12:34 | 8.6 | g/min | 13.1 | bar | 461.3 | g | Commencement of sampling 10-B.14 |
| 12:38 | 8.6 | g/min | 13.5 | bar | 495.7 | g | Commencement of sampling 10-B.15 |
| 12:42 | 8.6 | g/min | 13.3 | bar | 530 | g | Commencement of sampling 10-B.16 |
| | | | | | | | Commencement of sampling 10-B.17, flushed |
| 12:46 | 7.4 | g/min | 13.1 | bar | 7.4 | g | with MeOH (10 ml/min) |
| 12:50 | 7.9 | g/min | 13.3 | bar | 38.8 | g | Commencement of sampling 10-C.1 |
| 12:54 | | g/min | 13.1 | bar | 66.2 | g | Commencement of sampling 10-C.2 |
| 12:58 | 7.9 | g/min | 13.3 | bar | 97.8 | g | Commencement of sampling 10-C.3 |
| 13:02 | 7.7 | g/min | 13.3 | | 129 | | Commencement of sampling 10-C.4 |
| 13:06 | 7.8 | g/min | 13.3 | bar | 160.5 | g | Commencement of sampling 10-C.5 |
| 13:10 | 7.8 | g/min | 12.8 | bar | 192 | g | Commencement of sampling 10-C.6 |
| 13:14 | 7.9 | g/min | 13.3 | bar | 223.4 | g | Commencement of sampling 10-C.7 |
| 13:18 | 8.1 | g/min | 13.3 | bar | 254.9 | g | Commencement of sampling 10-C.8 |
| 13:22 | 7.9 | g/min | 13.3 | bar | 286.3 | g | Commencement of sampling 10-C.9 |
| 13:26 | 7.8 | g/min | 12.8 | bar | 317.5 | g | Commencement of sampling 10-C.10 |
| 13:30 | 7.8 | g/min | 13.3 | bar | 348.9 | g | Commencement of sampling 10-C.11 |
| 13:34 | 7.8 | g/min | 13 | bar | 380.2 | g | Commencement of sampling 10-C.12 |
| 13:38 | 7.7 | g/min | 13.1 | | 411.5 | | Commencement of sampling 10-C.13 |
| 13:42 | 7.8 | g/min | 13.1 | bar | 442.9 | g | Commencement of sampling 10-C.14 |
| 13:46 | 7.7 | g/min | 13.5 | bar | 474.1 | g | Commencement of sampling 10-C.15 |
| 13:50 | 7.7 | g/min | 13 | bar | 505.5 | g | Commencement of sampling 10-C.16 |
| 13:54 | 7.9 | g/min | 13.3 | bar | 536.9 | g | Commencement of sampling 10-C.17 |
| 13:58 | 7.8 | g/min | 12.5 | bar | 568.2 | g | Commencement of sampling 10-C.18 |
| 14:02 | 8 | g/min | 13 | bar | 599.7 | g | MeOH flush ended |
| 14:03 | 7.9 | g/min | 13 | bar | 607.6 | g | Valve flushed with MeOH via column |
| 14:08 | 3 | g/min | 13.1 | | 641.8 | | MeOH flush stopped |
| 14:10 | 0 | g/min | 4.5 | bar | 643.2 | g | Silica gel filling adjusted by ~2 mm to ~573 mm, |
| | | | | | | | for reduction of dead volume the feed was screwed |
| | | | | | | | downward |

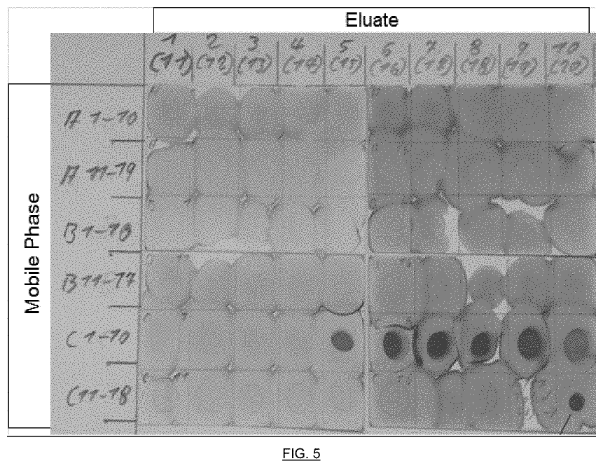

A small amount of the eluate Is applied to a silica gel plate (aluminium TIC foils 5×7.5 cm, silica gel 60 F 254), after it has dried off a 10% silver nitrate solution is trickled over, and it is dried again, in the subsequent irradiation with UV light, the catalyst or silver halide formed becomes visible as a brown spot and hence batches with a catalyst content become visible, see FIG. 5.

The catalyst-free eluates (10-A6 to 10-C3) were combined (1224.9 g) and concentrated under reduced pressure (80° C./1 mbar).

The catalyst (tri-n-butyl(2-hydroxyethyl)phosphonium bromide) was separable from the product mixture by the column chromatography via continuous chromatography (continuous product application). The GC purity of the product thus obtained rose from 96.8 area % to 97.9 area %; the HPLC purity rose from 93.6% to 98.1%; the phosphorus content fell from 0.319% to <10 ppm and the color number fell from 22 to 6.

The catalyst-containing eluates (10-C4 to 10-C11) were combined (255.9 g) and concentrated on a rotary evaporator (80° C./1 mbar), giving a pale yellowish liquid with white solids.

Phosphorus content by AAS or ³¹PNMR: 4.45% by weight. The sample additionally contains large amounts of glycerol carbonate methacrylate and the polar impurities such as glycerol monomethacrylate (hydrolysis product of the epoxide) and glycerol dimethacrylate (double crosslinker).

The silica gel used (160 g in dry form) gave 250 g of concentrated product in the first pass, in the repetition, in the second pass, 295 g of concentrated product were obtained (P content: <15 ppm), and in the third pass only 141 g of concentrated 2-oxo-1,3-dioxolan-4-yl)methyl methacrylate (P content: <15 ppm) were obtained before halides were detected in the product fraction. This suggests breakthrough (channel formation) in the column packing. After flushing of the column packing with methanol, flushing of the stationary phase with 3.0 usable volumes of water and adjustment of the stationary phase to first methanol and then toluene, it was possible to restore the original capacity. The cleaning with methanol does not seem to be sufficiently polar, and so the catalyst or conversion products thereof are flushed out of the stationary phase only inadequately with methanol.

### Example 10: Scale-Up of Example 8 to a 22.5 Mol (6 l) Batch

Apparatus:

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

3198.4 g (22.5 mol) glycidyl methacrylate

196.4 g solution of tri-n-butyl(2-hydroxyethyl)phosphonium bromide in acetonitrile

147.3 g (0.45 mol=2 mol % based on epoxide) tri-n-butyl(2-hydroxyethyl)phosphonium bromide

49.1 g acetonitrile

0.48 g (150 ppm based on epoxide) HQME stabilizer

5 bar CO₂

Procedure:

Glycidyl methacrylate and the HQME stabilizer were introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to ˜70° C. (circulation temperature: ˜60° C.). An exothermic reaction commences at ˜70° C., but is not very marked. The mixture is heated stepwise from 70 to 85° C. within 3 hours, and every temperature increase causes an exotherm in the tank. For further reaction, the mixture was heated stepwise to 90° C. After a reaction time of ˜35.5 h (reaction temperature of 70-90° C.), the reaction was ended. The mixture was ventilated and discharged. Yellow, slightly cloudy glycerol carbonate methacrylate (4369.5 g=98.9% of theory) was obtained.

Analysis:

| | |
|---|---|
| Pt/Co color number: | 53 |
| GC analysis: | |
| Glycidyl methacrylate | 0.57 GC area % |
| Glycerol carbonate | 0.04 GC area % |
| Glycerol trimethacrylate | 0.03 GC area % |
| Glycerol carbonate methacrylate | 90.9 GC area % |
| Glycerol dimethacrylates | 1.21 GC area % |
| Glycerol monomethacrylates | 0.85 GC area % |

In spite of performance of the reaction by the process according to the invention, the reaction cannot be scaled up again. The product is pale yellow in color, but does not meet the specification in the crosslinker category, in spite of a reaction time of 35.5 h, the mixture has additionally not been fully converted. The formation of the crosslinker with otherwise high quality again suggests an undersupply of CO₂. The diffusion of the CO₂ into the reaction phase is one possible cause, and so the reaction is to be heated up more slowly hereinafter in order to counteract the slow CO₂ supply by a Sower consumption.

Reaction scale increased once again with optimized CO₂ pressure, color number acceptable, but crosslinker and purity are inadequate. Not an example according to the present invention.

### Example 11: Phosphonium Bromide Catalyst 30 Mol Batch, Colder, Different Stirrer

Apparatus:

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

3695.9 g (26.0 mol) glycidyl methacrylate

226.9 g solution of tri-n-butyl(2-hydroxyethyl)phosphonium bromide in acetonitrile

170.2 g (0.52 mol=2 mol % based on epoxide) tri-n-butyl(2-hydroxyethyl)phosphonium bromide

56.7 g acetonitrile

0.554 g (150 ppm based on epoxide) HQME stabilizer

5 bar CO₂

Procedure:

Glycidyl methacrylate and the HQME stabilizer were introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to ˜50° C. An exothermic reaction commences during the dwell time at 50° C., but is not very marked (˜56° C.). The mixture is heated stepwise to 75° C. within six hours: no exothermicity is observed here. For further reaction, the mixture was heated stepwise to 80° C. After a reaction time of ˜28 h, the reaction was ended. The mixture was ventilated and discharged. Yellow, slightly cloudy glycerol carbonate methacrylate (5086.9 g=98.9% of theory) was obtained.

Analysis:

| | Crude product | Degassed |
|---|---|---|
| Pt/Co color number: | 23 | n.d. |
| GC analysis: | | |
| Acetonitrile | 2.59 GC area % | 0.15 GC area % |
| Glycidyl methacrylate | 0.07 GC area % | 0.07 GC area % |
| Glycerol carbonate | n.m. | n.m. |
| Glycerol trimethacrylate | 0.10 GC area % | 0.13 GC area % |
| Glycerol carbonate methacrylate | 95.2 GC area % | 97.3 GC area % |
| Glycerol dimethacrylates | 0.20 GC area % | 0.24 GC area % |
| Glycerol monomethacrylates | 0.41 GC area % | 0.42 GC area % |

Solely by virtue of the temperature regime at the start of the reaction, it is now also possible to Implement the reaction on a larger scale. When it is ensured particularly in the initial period of the reaction that the reaction is sufficiently slow to fake account of the CO₂ supply, crosslinkers can be distinctly reduced. Since the reaction is highly dependent on the CO₂ gas supply, the use of a gas-aspirating stirrer or of a reaction tank sparged from beneath is an option.

Reaction scale increased once again with optimized CO₂ pressure and stepwise increase in reaction temperature, color number, crosslinker and purity are very good. Example according to the present invention.

### Example 12: Removal and Reuse of the Phosphonium Bromide Catalyst; Collapse in Selectivity and Activity

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

3695.9 g (26.0 mol) glycidyl methacrylate

226.9 g solution of tri-n-butyl(2-hydroxyethyl)phosphonium bromide in acetonitrile

170.2 g (0.52 mol=2 mol % based on epoxide) tri-n-butyl(2-hydroxyethyl)phosphonium bromide

56.7 g acetonitrile

0.554 g (150 ppm based on epoxide) HQME stabilizer

5 bar CO₂

Procedure:

Glycidyl methacrylate and the HQME stabilizer were introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to 50° C. An exothermic reaction commences during the dwell time at 50° C., but is not very marked (56° C.). The mixture is heated stepwise to 75° C. within six hours; no exothermicity Is observed here. For further reaction, the mixture was heated stepwise to 80° C. After a reaction time of 28 h, the reaction was ended. The mixture was ventilated and discharged. The crude ester obtained was examined by means of ³¹P NMR and AAS/ICP-MS for its catalyst content and then worked up according to Example 9 in order to isolate the catalyst. Subsequently, the reaction was repeated with the isolated catalyst.

Analysis:

| Cat. recycling | Orig. | 1. | 2 | 3 | 4 | |
|---|---|---|---|---|---|---|
| Phosphorus content (AAS) [w %] | | 0.32 | 0.31 | 0.31 | 0.30 | 0.30 |
| Halide content (titration) [w %] | 0.84 | 0.80 | 0.75 | 0.68 | 0.59 | |
| Pt/Co color number: | 22 | 24 | 36 | 43 | 73 | |
| GC analysis: | | | | | | |
| Glycidyl methacrylate (GC area %) | 0.02 | 0.01 | 0.18 | 0.87 | 1.8 | |
| Glycerol carbonate (GC area %) | n.m. | n.m. | n.m. | n.m. | n.m. | |
| Glycerol trimethacrylate (GC area %) | 0.03 | 0.02. | 0.02 | 0.02 | 0.02 | |
| Glycerol carbonate methacrylate (GC area %) | 98.2 | 96.6 | 96.5 | 94.2 | 92.1 | |
| Glycerol dimethacrylates (GC area %) | 0.28 | 0.87 | 0.84 | 0.93 | 0.89 | |
| Glycerol monemethacrylates (GC area %) | 0.05 | 0.58 | 0.86 | 1.3 | 1.5 | |

Analogously to Example 9, there is at first (in the first experiment) a rise in the purity of the product as a result of purification using silica gel. However, as a result of the reuse of the catalyst, the polar impurities are transferred into the subsequent batch again with the catalyst, and so the product quality declines back to a purity correspondingly without chromatography, or the crude product. From about the third recycling of the catalyst, a distinct drop commences in conversion and selectivity. At the same time, there is a drop in the value for soluble halides determined via precipitative titration. Therefore, there appears to be a creeping loss of halides, presumably via organically bound bromide. The bromide loss is to be compensated for again hereinafter by addition of ammonium bromides. Since alkylammonium bromides would remain permanently in the catalyst solution, one option is the use of ammonium bromide, which could merely cause nitrogen contamination in the product but does not dilute the catalyst in a sustained manner with extraneous salts.

The composition of a catalyst solution, after isolating the catalyst using silica gel, corresponds roughly to:

| | |
|---|---|
| Glycidyl methacrylate (GC area %) | 1.20 |
| Glycerol trimethacrylate (GC area %) | n.m. |
| Glycerol carbonate methacrylate (GC area %) | 27.2 |
| Glycerol dimethacrylates (GC area %) | 0.40 |
| Glycerol monomethacrylates (GC area %) | 5.20 |
| Catalyst (³¹P/¹H-NMR or AAS) | 67.4 % by wt. |

Reaction scale increased once again with optimized CO₂ pressure and stepwise increase in reaction temperature, color number, crosslinker and purity are at first very good. As recycling of the catalyst continues, there is a collapse in color number, conversion and purity. Not an example according to the present invention.

### Example 13: Removal and Reuse of the Phosphonium Bromide Catalyst; Retention of Selectivity and Activity by Adjustment of the Halide Content

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

3695.9 g (26.0 mol) glycidyl methacrylate

226.9 g solution of tri-n-butyl(2-hydroxyethyl)phosphonium bromide in acetonitrile

170.2 g (0.52 mol=2 mol % based on epoxide) tri-n-butyl(2-hydroxyethyl)phosphonium bromide

56.7 g acetonitrile

0.554 g (150 ppm based on epoxide) HQME stabilizer

5 bar CO₂

Procedure:

Glycidyl methacrylate and the HQME stabilizer were introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to ˜50° C. An exothermic reaction commences during the dwell time at 50° C., but is not very marked (˜56° C.). The mixture is heated stepwise to 75° C. within six hours; no exothermicity is observed here. For further reaction, the mixture was heated stepwise to 80° C. After a reaction time of ˜28 h, the reaction was ended. The mixture was ventilated and discharged. The crude ester obtained was examined by means of ³¹P NMR and AAS for its phosphorus content. By means of precipitative titration according to Mohr, the soluble bromides were determined. Subsequently, the crude ester was worked up according to Example 9 in order to isolate the catalyst. The isolated catalyst solution was adjusted to the necessary stoichiometry relative to the phosphorus content by addition of ammonium bromide, and the catalyst solution thus obtained was used in a subsequent experiment.

Analysis:

| Cat. recycling | Orig. | 1. | 2 | 3 | 4 | 5 | 6 |
|---|---|---|---|---|---|---|---|
| Phosphorus content (AAS) [w %] | 0.32 | 0.31 | 0.31 | 0.30 | 0.30 | 0.30 | 0.30 |
| Halide content (titration) [w %] | 0.84 | 0.82 | 0.82 | 0.80 | 0.80 | 0.80 | 0.80 |
| Pt/Co color number: | 22 | 24 | 25 | 24 | 26 | 28 | 25 |
| GC analysis: | | | | | | | |
| Glycidyl methacrylate (GC a %) | 0.02 | 0.05 | 0.04 | 0.09 | 0.07 | 0.11 | 0.09 |
| Glycerol carbonate (GC a %) | n.m. | n.m. | n.m. | n.m. | n.m. | n.m. | n.m. |
| Glycerol trimethacrylate (GC a %) | 0.03 | 0.02. | 0.02 | 0.03 | 0.02 | 0.02 | 0.03 |
| Glycerol carb. methacrylate (GC a %) | 98.2 | 96.6 | 96.5 | 96.7 | 96.3 | 96.1 | 96.4 |
| Glycerol dimethacrylates (GO a %) | 0.28 | 0.87 | 0.84 | 0.85 | 0.89 | 0.91 | 0.85 |
| Glycerol monomethacrylates (GO a %) | 0.05 | 0.58 | 0.64 | 0.72 | 0.68 | 0.65 | 0.71 |

Reaction scale increased once again with optimized CO₂ pressure and stepwise increase in reaction temperature, color number, crosslinker and purity are at first very good and remain so even after repeated catalyst recycling. Example according to the present invention.

### Example 14: Tributylphosphonium Bromide Catalyst with Ideal Reaction Parameters Using the Example of Isobutene Oxide

Apparatus:

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

2882.4 g (40.0 mol) isobutene oxide

348.2 g solution of tri-n-butyl(2-hydroxyethyl)phosphonium bromide in acetonitrile

261.8 g (0.8 mol=2 mol % based on epoxide) tri-n-butyl(2-hydroxyethyl)phosphonium bromide 86.4 g acetonitrile

5 bar CO₂

Procedure:

isobutene oxide was introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to ˜50° C. An exothermic reaction commences during the dwell time at 50° C., but is not very marked. The mixture is heated stepwise to 75° C. within six hours. For further reaction, the mixture was heated stepwise to 80° C. After a reaction time of ˜28 h, the reaction was ended. The mixture was ventilated and discharged. Colorless isobutyl carbonate (4,4-dimethyl-1,3-dioxolan-2-one) (4974 g=99.6% of theory) was obtained.

Analysis:

| | Crude product | Degassed |
|---|---|---|
| Pt/Co color number: | 8 | 8 |
| GC analysis: | | |
| Acetonitrile | 2.59 GC area % | 0.02 |
| Isobutene oxide | 0.07 GC area % | 0.07 |
| Isobutyl carbonate | 97.3 GC area % | 99.8 |

Contamination by the catalyst is not visible in the GC owing to the non-volatility of the catalyst salt. The actual purity is thus lower.

Excellent reaction with >99% yield and excellent color number. The product is considerably less demanding than glycerol carbonate methacrylate. The process can be applied to other epoxides without difficulty. Example according to the present invention.

### Example 15: Catalyst Recycling Using the Example of Isobutene Oxide

6.4 ltr. reaction tank/pressure vessel with base outlet tap, manometer [0-40 bar], pressure sensor with data recording, pressure relief valve, propeller stirrer, NiCrNi thermocouple with data recording, PT100 thermocouple [internal temperature control], 2× inlet with tap [CO₂ introduction, ventilation], inlet/riser tube with tap [135 mm under lid, catalyst addition/sampling], stirrer motor [with speed control and off switch in the event of rising viscosity], cold thermostat [temperature control via internal tank temperature], CO₂ valves [max. 20 l/min=about <8 bar, non-return valve], balance [data recording for CO₂ consumption], HPLC pump [with 50 ml TI pump head] for catalyst dosage

Batch:

2882.4 g (40.0 mol) isobutene oxide

348.2 g solution of tri-n-butyl(2-hydroxyethyl)phosphonium bromide in acetonitrile

261.8 g (0.8 mol=2 mol % based on epoxide) tri-n-butyl(2-hydroxyethyl)phosphonium bromide

86.4 g acetonitrile

5 bar CO₂

Procedure:

Isobutene oxide was introduced into the tank, which was closed and stirred (125 rpm). The tank was pressurized with CO₂ to 5 bar and opened towards the CO₂ reservoir in order to keep the pressure constant at 5 bar. The catalyst solution was added to the tank via the riser tube with an HPLC pump and the conduits were flushed with acetonitrile once more into the tank. The reaction mixture was heated to ˜50° C. An exothermic reaction commences during the dwell time at 50° C., but is not very marked. The mixture is heated stepwise to 75° C. within six hours. For further reaction, the mixture was heated stepwise to 80° C. After a reaction time of ˜28 h, the reaction was ended. The mixture was ventilated and discharged. Colorless isobutyl carbonate was obtained, which was worked up according to Example 9 in order to isolate the catalyst. The isolated catalyst solution was examined by means of ³¹P NMR and AAS for its phosphorus content (0.5% by weight). By means of precipitative titration according to Mohr, the soluble bromides (1.28% by weight) were determined and these were adjusted to the calculated stoichiometry relative to the phosphorus content by addition of ammonium bromide after each experiment. The catalyst thus obtained was reused as catalyst for a subsequent experiment.

Analysis:

| | | | | | | | |
|---|---|---|---|---|---|---|---|
| Cat. recycling | Orig. | 1. | 2 | 3 | 4 | 5 | 6 |
| Pt/Co color number: | 8 | 7 | 9 | 12 | 9 | 11 | 10 |

GC analysis of the degassed and catalyst-free product:

| | | | | | | | |
|---|---|---|---|---|---|---|---|
| Acetonitrile [GC area %] | 0.02 | 0.03 | 0.02 | 0.04 | 0.03 | 0.03 | 0.06 |
| Isobutene oxide [GC area %] | 0.07 | 0.07 | 0.09 | 0.08 | 0.12 | 0.08 | 0.09 |
| Isobutyl carbonate [GC area %] | 99.8 | 99.6 | 99.7 | 99.5 | 99.8 | 99.6 | 99.4 |

Excellent reaction with >99% yield and excellent color number. The product is considerably less demanding than glycerol carbonate methacrylate even with repeated catalyst recycling. The process can again be applied to other epoxides without difficulty. Example according to the present invention.

### Preferred Items

Item 1. Process for preparing cyclic organic carbonates, characterized in that the molar ratio of CO₂ to catalyst is >0.01 before the epoxide is converted.
Item 2. Process for preparing cyclic organic carbonates according to item 1, characterized in that an epoxide is initially charged in the presence of CO₂ and then a catalyst is added.
Item 3. Process for preparing cyclic organic carbonates according to Item 1, characterized in that the reaction scale is greater than 5 mol.
Item 4. Process for preparing cyclic organic carbonates according to item 1, characterized in that the reaction temperature is below SOX.
Item 5. Process for preparing cyclic organic carbonates according to item 4, characterized in that the temperature is increased stepwise.
Item 6. Process for preparing glycerol carbonate (meth)acrylate, characterized in that a glycidyl(meth)acrylate is initially charged in the presence of CO₂ and then the catalyst is added.
Item 7. Process for preparing glycerol carbonate (meth)acrylate according to item 6, characterized in that the reaction temperature is below 90° C.
Item 8. Process for preparing cyclic organic carbonates according to any of items 1-5, characterized in that the partial pressure of the CO₂ is between 1-10 bar, preferably 2-8 bar and more preferably between 3 and 7 bar.
Item 9. Process for preparing cyclic organic carbonates according to any of Items 1-5, characterized in that the catalyst is selected from the group of the trialkylhydroxyalkylphosphonium bromides and trialkylhydroxyalkylammonium halides, preferably trialkylhydroxyalkylammonium bromide, more preferably tributylhydroxyethylphosphonium bromide.
Item 10. Process for preparing cyclic organic carbonates according to any of Items 1-5, characterized in that the catalyst is isolated from the reaction mixture.
Item 11. Process for preparing cyclic organic carbonates according to Item 10, characterized in that the catalyst is supplied to at least one further reaction.
Item 12. Process for preparing cyclic organic carbonates according to any of Items 10-11, characterized in that the halide content is adjusted to the original stoichiometry by adding a soluble halide salt.
Item 13. Process for preparing cyclic organic carbonates according to any of Items 10-12, characterized in that the halide content is adjusted to the original stoichiometry by adding a soluble halide salt and is supplied to at least one further reaction.
Item 14. Process for preparing cyclic organic carbonates according to any of Items 10-13, characterized in that the catalyst is reactivated by adding bromide salts selected from the group of ammonium bromide, alkylphosphonium bromides, hydroxyalkylammonium bromides, hydroxyalkylphosphonium bromides, alkylsulfonium bromides.
Item 15. Process for removing a catalyst salt, characterized in that the polarity of the product solution is lowered by adding a solvent to such a degree that the catalyst salt is absorbed by filtering through a polar stationary phase, and hence the product is freed continuously from the catalyst.
Item 16. Cyclic organic carbonates prepared according to Items 1-14 with a color number of the product of <500, more preferably <100, more preferably <50.
Item 17. Cyclic organic carbonates prepared according to items 1-14 with a concentration of unsaturated epoxides in the end product of less than 1000 ppm.
Item 18. Cyclic organic carbonates prepared according to Items 1-14 with a content of dimethacrylate by-products in the end product of less than 1% by weight.

1-15. (canceled)

16. A process for preparing cyclic organic carbonates, comprising:
a) charging a reactor with an epoxide in the presence of CO₂;
b) after step a), adding a catalyst;
wherein the reaction scale is greater than 5 mol.

17. The process of claim 16, wherein the molar ratio of CO₂ to catalyst is >0.01 before the epoxide is converted.

18. The process of claim 16, wherein the cyclic organic carbonate is glycerol carbonate (meth)acrylate and the epoxide is glycidyl (meth)acrylate.

19. The process of claim 16, wherein the reaction temperature is below 90° C.

20. The process of claim 16, wherein the reaction temperature is between 10° C. and 85° C.

21. The process of claim 16, wherein the temperature is increased stepwise.

22. The process of claim 16, wherein the CO₂ insertion is effected at pressures between 1 and 10 bar.

23. The process of claim 16, wherein the catalyst is selected from the group consisting of: trialkylhydroxyalkylphosphonium bromides and trialkylhydroxyalkylphosphonium halides.

24. The process of claim 16, wherein the catalyst content of the reaction mixture is between 0.05 mol % and 25 mol %.

25. The process of claim 16, wherein the catalyst is isolated from the reaction mixture.

26. The process of claim 25, wherein the polarity of the product solution is lowered by adding a solvent to such a degree that the catalyst salt is absorbed by filtering through a polar stationary phase, and hence the product is freed continuously from the catalyst.

27. The process claim 26, wherein the catalyst is reactivated by adding bromide salts selected from the group of ammonium bromide, alkylphosphonium bromides, hydroxyalkylammonium bromides, hydroxyalkylphosphonium bromides, alkyl sulfonium bromides.

28. The process of claim 16, wherein the epoxide is reacted with the CO₂ in the presence of at least one stabilizer selected from the group consisting of: phenothiazine, tempo, tempol and mixtures thereof is used.

29. The process of claim 16, wherein the epoxide is reacted with the CO₂ in the presence of at least one stabilizer, wherein said stabilizer is a substituted phenol derivative.

30. The process of claim 29, wherein the stabilizer content is between 20 ppm and 700 ppm.

31. The process of claim 22, wherein the reaction temperature is between 20° C. and 70° C.

32. The process of claim 31, wherein the catalyst is tributylhydroxyethylphosphonium bromide.

33. The process of claim 31, wherein the catalyst content of the reaction mixture is between 0.05 mol % and 25 mol %.

34. The process of claim 33, wherein the polarity of the product solution is lowered by adding a solvent to such a degree that the catalyst salt is absorbed by filtering through a polar stationary phase, and hence the product is freed continuously from the catalyst.

35. The process of claim 31, wherein the epoxide is reacted with the CO₂ in the presence of at least one stabilizer, wherein said stabilizer is a substituted phenol derivative.
